(19) United States
(12) Patent Application Publication
(10) Pub. No.: US 2023/0203589 A1
(43) Pub. Date: Jun. 29, 2023

(54) PERSONALISED RECOMMENDED DAILY INTAKE FOR NUTRIENTS BASED ON INDIVIDUAL GENETIC RISK SCORES

(71) Applicant: SOCIETE DES PRODUITS NESTLE S.A., Vevey (CH)

(72) Inventors: JORG HAGER, Houtaud (FR); AKIKO KUMAGAI, Tokyo (JP)

(21) Appl. No.: 17/999,137

(22) PCT Filed: May 20, 2021
(86) PCT No.: PCT/EP2021/063416

§ 371 Date: Nov. 17, 2022

(30) Foreign Application Priority Data
May 20, 2020 (EP) 20175634.3

(51) Int. Cl. C12Q 1/6883

(52) U.S. Cl. CPC C12Q 1/6883; C12Q 2600/106; C12Q 2600/156

(57) ABSTRACT

The invention relates to systems and methods for determining personalized recommended daily intake, for example, the recommended daily allowance (RDA) for nutrients based on genetic information of the individual. In several embodiments, the systems and methods of the invention can be used to determine recommendations for specific nutrient supplements, foods, beverages, meals, menus, diets and recipes for an individual which are more accurately adapted to their needs based on their personal genetic risk scores.

### FIELD OF THE INVENTION

The invention relates to systems and methods for determining personalized recommended daily intakes, for example, the recommended daily allowance (RDA) for nutrients based on genetic information of the individual. In several embodiments, the systems and methods of the invention can be used to determine recommendations for specific nutrient supplements, foods, beverages, meals, diets, menus and recipes for an individual which are more accurately adapted to their needs based on their personal genetic risk scores.

### BACKGROUND TO THE INVENTION

A sufficient supply of nutrients is essential to ensure the maintenance of health in an individual. In order to estimate the amount of nutrients that a person needs on average to maintain their health, regulatory authorities in most countries have established dietary reference values (DRV) or recommended dietary allowance (RDA) for nutrients. RDAs are averaged values based on dietary intake data in an assumedly healthy population. However, they do not provide recommendations for nutrient intake thresholds for a specific individual. Indeed, the European Food Safety Authority (EFSA) explicitly states that DRVs are not nutrient goals or recommendations for individuals, but rather for populations.

In order to provide more accurate nutrient recommendations for individuals, other factors need to be taken into account. In particular, genetic influences may affect the variation of how nutrients are absorbed and metabolized by the body by particular individuals. At present, there is no accurate, non-invasive way to determine an individual's nutrient status and couple this with their individual nutrient needs. Certain nutrients such as vitamins and minerals can be measured through blood tests but these tests only provide a snapshot view of an individual's nutrient status at a specific time point but not their general propensity to have a nutrient deficiency based on their genetic predisposition.

The present invention provides an improvement on the general recommended dietary allowance (RDAs) of specific nutrients that are recommendations for population groups and are typically set by national regulatory authorities. In several embodiments of the invention, the systems and methods of the invention provide nutrient recommendations which are personalized for an individual user by taking into account individual genetic variation determined by their polygenic risk score profile.

### SUMMARY OF THE INVENTION

Genetic variants influence how nutrients are absorbed and metabolized by the body. Hundreds of genetic variants influence nutrient status in a person. The genetic variants which are single nucleotide polymorphisms (SNPs) of specific alleles are associated with a change in nutrient status that can be determined.

Knowing the effect size for each SNP of each allele, one can establish a polygenic risk score that summarizes the cumulative effects of all alleles present in an individual for a given nutrient, for example, vitamins or minerals levels. This genetic effect can then be taken into consideration when calculating an individual's nutrient requirements and provide recommendations for the intake of that nutrient for an individual thus establishing a personalized RDA based on the individual's genetic profile.

The present invention provides a solution in several embodiments of systems and methods, including algorithmic calculations of how to translate polygenic risk scores into quantitative recommendations for an individual's nutrient needs.

For many nutrients, the effect sizes of single genetic variants are small and may not allow useful recommendations for nutrient intake for an individual beyond the RDA. The levels of most nutrients, such as vitamins, are influenced by multiple genetic variants. For example, Vitamin B12 has thirty genetic variants influencing its concentration in blood. Therefore, the assessment for those nutrients with multiple genetic variants may be complex.

Polygenic risk scores are a powerful way to determine the combined effect size of all genetic variants influencing a trait. For example, in medical applications, polygenic risk scores are used to determine an individual's propensity to develop a disease. In nutrition, polygenic risk scores are rarely used.

In several embodiments of the invention, systems and methods for transforming genetic information related to specific genetic variants on alleles associated with nutrient traits are combined into a polygenic risk score.

In several embodiments of the invention, systems and methods for determining the polygenic risk scores can be used to adjust the RDAs for an individual for a plurality of nutrients.

In several embodiments of the invention, systems and methods for transforming the polygenic risk scores for various nutrients can be used to determine recommendations for specific nutrient supplements, foods, beverages, meals, diets, menus and recipes for an individual which are more accurately adapted to their improve or maintain their nutritional health.

### DESCRIPTION OF FIGURES

FIG. 1—Representative computer-implemented system of the invention

FIG. 2—Representative recommendation system of the invention

### DETAILED DESCRIPTION OF THE INVENTION

Dietary Reference Intake (DRI)

Dietary Reference Intake (DRI) is a system of nutrition recommendations from the Institute of Medicine (IOM) of the National Academies (United States) introduced in 1997 in order to broaden the existing guidelines known as Recommended Dietary Allowances (RDAs).

Recommended Daily Allowance (RDA)

Recommended Dietary Allowances (RDA) is the daily dietary intake level of a nutrient considered sufficient to meet the requirements of 97.5% of healthy individuals in each life-stage and sex group. The definition implies that the intake level would cause a harmful nutrient deficiency in just 2.5%. It is calculated based on the Estimated Average Requirements (EAR) and is usually approximately 20% higher than the EAR.

If the standard deviation (SD) of the EAR is available and the requirement for the nutrient is symmetrically distributed, the RDA is set at two SDs above the EAR:

RDA=EAR+2SD(EAR)

If data about variability in requirements are insufficient to calculate an SD, a coefficient of variation (CV) for the EAR of 10 percent is assumed, unless available data indicate a greater variation in requirements. If 10 percent is assumed to be the CV, then twice that amount when added to the EAR is defined as equal to the RDA. The resulting equation for the RDA is:

RDA=1.2(EAR)

This level of intake statistically represents 97.5 percent of the requirements of the population.

Estimated Average Requirements (EAR)

Estimated Average Requirements (EAR) for nutrients are calculated to satisfy the needs of 50% of the people in a specific age group based on a review of the scientific literature.

Adequate Intake (AI)

Adequate Intake (AI) for nutrients is the amount when no RDA has been established and it is based on what is considered to be adequate for a specific demographic group.

Tolerable Upper Intake Levels (UL)

Tolerable upper intake levels (UL), to caution against excessive intake of nutrients (such as fat soluble vitamins) that can be harmful in large amounts. This is the highest level of daily nutrient consumption that is considered to be safe for, and cause no side effects in, 97.5% of healthy individuals in each life-stage and sex group. The definition implies that the intake level would cause a harmful nutrient excess in just 2.5%.

Different national and regional authorities have different dietary reference values. For example, The European Food Safety Authority (EFSA) refers to the collective set of information as Dietary Reference Values (DRV), with Population Reference Intake (PRI) instead of RDA, and Average Requirement instead of EAR. AI and UL defined the same as in United States, but values may differ.

Reference RDAs

The reference RDAs generally are given per age and gender of an individual in a particular population.

For example, Table 1 demonstrates the RDAs for a 44 year old male which would be considered the “reference RDAs” for a 44 year old male not taking into account any individual genetic risk score per nutrient.

TABLE 1
Representative Recommended Daily Allowance for Male, 44 years old

| | | Highest | | | |
|---|---|---|---|---|---|
| Nutrient | EAR | RDA/AI | UL | Unit | Food sources, 100 grams |
| Vitamin A | 625 | 900 | 3000 | μg | cod liver oil, liver, dehydrated red sweet |
| | | | | | peppers, veal, dehydrated carrots |
| Thiamin (B₁) | 1.0 | 1.2 | ND | mg | fortified breakfast cereals, energy |
| | | | | | bars, vegetarian, and baby |
| | | | | | food products |
| Riboflavin (B₂) | 1.1 | 1.3 | ND | mg | fortified food products, lamb |
| | | | | | liver, spirulina |
| Niacin (B₃) | 12 | 16 | 35 | mg | fortified food products, baker's |
| | | | | | yeast, rice bran, instant coffee, fortified |
| | | | | | beverages |
| Pantothenic | NE | 5 | ND | mg | fortified food and beverage products, |
| acid (B₅) | | | | | dried shiitake mushrooms, beef liver, |
| | | | | | rice bran |
| Vitamin B₆ | 1.1 | 1.3 | 100 | mg | fortified food and beverage products, |
| | | | | | rice bran, fortified margarines, |
| | | | | | ground sage |
| Biotin (B₇) | NE | 30 | ND | μg | organ meats, eggs, fish, meat, seeds, |
| | | | | | nuts^{[9]} |
| Folate (B₉) | 320 | 400 | 1000 | μg | baker's yeast, fortified food and |
| | | | | | beverage products, poultry liver |
| Cobalamin | 2.0 | 2.4 | ND | μg | shellfish, beef, animal liver, fortified food |
| (B12) | | | | | and beverage products |
| Vitamin C | 75 | 90 | 2000 | mg | fortified beverages, dried sweet |
| | | | | | peppers, raw acerola, |
| | | | | | dried chives and coriander, rose hips, |
| | | | | | fortified food products |
| Vitamin D | 10 | 15 | 100 | μg | cod liver oil, mushrooms (if exposed |
| | | | | | to ultraviolet light), halibut, mackerel, |
| | | | | | canned sockeye salmon |
| α- | 12 | 15 | 1000 | mg | wheat germ oil, fortified food and |
| tocopherol | | | | | beverage products, hazelnut oil, |
| (Vitamin E) | | | | | fortified peanut butter, chili powder |
| Vitamin K | NE | 120 | ND | μg | dried spices, fresh parsley, cooked and |
| | | | | | raw kale, chard, other leaf vegetables |
| Choline | NE | 550 | 3500 | mg | egg yolk, organ meats from beef |
| | | | | | and pork, soybean oil, fish roe |
| Calcium | 800 | 1000 | 2500 | mg | fortified cereals, |
| | | | | | beverages, tofu, energy bars, and baby |
| | | | | | foods, dried basil and other spices, |
| | | | | | dried whey, cheese, milk powder |
| Chloride | NE | 2300 | 3600 | mg | table salt |
| Chromium | NE | 35 | ND | μg | broccoli, turkey ham, |
| | | | | | dried apricots, tuna, pineapple, grape |
| | | | | | juice^{[10]} |
| Copper | 700 | 900 | 10000 | μg | animal liver, seaweed products, |
| | | | | | dried shiitake |
| | | | | | mushrooms, oysters, sesame |
| | | | | | seeds, cocoa |
| | | | | | powder, cashews, sunflower seeds |
| Fluoride | NE | 4 | 10 | mg | public drinking water, |
| | | | | | where fluoridation is performed or |
| | | | | | natural fluorides are present, |
| | | | | | tea, raisins |
| Iodine | 95 | 150 | 1100 | μg | iodized salt, kelp, cod |
| Iron | 6 | 8 | 45 | mg | dried thyme and other spices, fortified |
| | | | | | foods, including baby foods, animal |
| | | | | | organ meats |
| Magnesium | 350 | 420 | 350 | mg | crude rice bran, cottonseed flour, hemp |
| | | | | | seeds, dried spices, cocoa powder, |
| | | | | | fortified beverages |
| Manganese | NE | 2.3 | 11 | mg | fortified beverages and infant formulas, |
| | | | | | ground cloves and other dried |
| | | | | | spices, chickpeas, fortified breakfast |
| | | | | | cereals |
| Molybdenum | 34 | 45 | 2000 | μg | legumes, grain |
| | | | | | products, nuts and seeds^{[11]} |
| Phosphorus | 580 | 700 | 4000 | mg | baking powder, instant |
| | | | | | pudding, cottonseed meal, hemp seeds, |
| | | | | | fortified beverages, dried whey |
| Potassium | NE | 3400 | ND | mg | baking powder, dried parsley and other |
| | | | | | spices, instant tea and instant coffee, |
| | | | | | dried tomatoes, dried sweet |
| | | | | | peppers, soy sauce |
| Selenium | 45 | 55 | 400 | μg | Brazil nuts and mixed nuts, |
| | | | | | animal kidneys, dried eggs, oysters, |
| | | | | | dried cod |
| Sodium | NE | 1500 | 2300 | mg | table salt, baking soda, soup bouillon |
| | | | | | cube, seasoning mixes, onion |
| | | | | | soup mix, fish sauce |
| Zinc | 9.4 | 11 | 40 | mg | oysters, fortified breakfast cereals, baby |
| | | | | | foods, beverages, peanut butter, and |
| | | | | | energy bars, wheat germ |

NE: EARs have not yet been established or not yet evaluated; ND: ULs could not be determined, and it is recommended that intake from these nutrients be from food only, to prevent adverse effects.

Personalised RDA

“Personalised RDA” or “individualized RDA” refers to a recommended daily allowance of a specific nutrient which has been customized for an individual based on their polygenic risk score for that nutrient.

The personalized RDA may be compared to the reference RDA to determine whether the individual need for a particular nutrient is below average, average or above average compared to the reference RDA for that nutrient.

DNA Sample for SNP Genotyping

In general, the term “sample” as used herein refers to any body fluid or other tissue sample types, e.g. blood, plasma, serum, sputum, saliva, sweat (perspiration) or urine. Techniques for obtaining such samples from subjects are well known. The term also includes samples of other tissues or fluids obtained by contact with body tissues, e.g. exhaled breath or contact with the skin.

A sample of an individual's DNA can be analysed from a biological sample from any body fluid or tissue listed above. In a preferred embodiment of the invention, the DNA sample is from a buccal swab. From this DNA sample, the individual's SNP genotype profile can measure genetic variations of single nucleotide polymorphisms (SNPs) between individuals and to determine the polygenic risk score per nutrient for an individual.

Single Nucleotide Polymorphism

A single nucleotide polymorphism (SNP) represents a difference in a single nucleotide on an allele of a gene. More than 335 million SNPs have been found across humans from multiple populations. Variations in the DNA sequences of individuals affect how they may differently develop diseases, respond to pathogens, chemicals, drugs, vaccines, and other agents. SNPs are also critical for personalized nutrition such as metabolic response to nutrients.

In several embodiments of the invention, reference SNP databases may be queried in the systems and methods of the invention in order to compare the individual's SNP profile with the reference SNP database. Some reference SNP databases include the following:

dbSNP is a SNP database from the National Center for Biotechnology Information (NCBI);

Kaviar is a compendium of SNPs from multiple data sources including dbSNP;

SNPedia is a wiki-style database supporting personal genome annotation, interpretation and analysis;

OMIM is a database which describes the association between polymorphisms and diseases;

dbSAP is a single amino-acid polymorphism database for protein variation detection;

Human Gene Mutation Database provides gene mutations causing or associated with human inherited diseases and functional SNPs;

International HapMap Project is where researchers are identifying Tag SNPs to be able to determine the collection of haplotypes present in each subject; and

GWAS Central allows users to visually interrogate the actual summary-level association data in one or more genome-wide association studies.

Polygenic Risk Score (PGS)

A polygenic risk score, also known as a genetic risk score, or genome-wide score, is a number based on variation in multiple genetic loci and their associated weights. It serves as the best prediction for a trait. In the present invention, the trait of interest is a particular nutrient recommended daily allowance that can be made when taking into account variation in multiple genetic variants.

A polygenic score (PGS) is constructed from the “weights” derived from a genome-wide association study (GWAS). In a GWAS, a set of genetic markers, usually SNPs, is genotyped on a training sample, and effect sizes are estimated for each marker's association with the trait of interest. These weights are then used to assign individualized polygenic scores in an independent replication sample.

There are various methodologies that can be used to generate the weights of the SNPs and how to determine which SNPs should be included.

The simplest so-called “naïve” method of construction sets weights equal to the coefficient estimates from a regression of the trait on each genetic variant. The included SNPs may be selected using an algorithm that attempts to ensure that each marker is approximately independent. Failing to account for non-random association of genetic variants will typically reduce the score's predictive accuracy. This is important because genetic variants are often correlated with other nearby variants, such that the weight of a causal variant will be attenuated if it is more strongly correlated with its neighbors than a null variant. This is called linkage disequilibrium, a common phenomenon that arises from the shared evolutionary history of neighboring genetic variants. Further restriction can be achieved by multiple-testing different sets of SNPs selected at various thresholds, such as all SNPs which are genome-wide statistically-significant hits or all SNPs p<0.05 or all SNPs with p<0.50, and the one with greatest performance used for further analysis; especially for highly polygenic traits, the best polygenic score will tend to use most or all SNPs.

The Bayesian method accounts for the distribution of effect sizes to improve the accuracy of a polygenic score. One of the most popular modern Bayesian methods uses “linkage disequilibrium prediction” (LDpred for short) to set the weight for each SNP equal to the average of its posterior distribution after linkage disequilibrium has been accounted for. LDpred tends to outperform simpler methods of pruning and thresholding, especially for large sample sizes.

Penalized regression methods, such as LASSO and ridge regression, can also be used to improve the accuracy of polygenic scores. Penalized regression can be interpreted as placing informative prior probabilities on how many genetic variants are expected to affect a trait, and the distribution of their effect sizes. In other words, these methods in effect “penalize” the large coefficients in a regression model and shrink them conservatively. Ridge regression accomplishes this by shrinking the prediction with a term that penalizes the sum of the squared coefficients. LASSO accomplishes something similar by penalizing the sum of absolute coefficients.

Any of the above methods may be used to calculate the polygenic risk score for a particular nutrient.

Nutrients

The term “nutrient” refers to compounds having a beneficial effect on the body e.g. to provide energy, growth or health. The term includes organic and inorganic compounds.

As used herein the term nutrient may include, for example, macronutrients, micronutrients, essential nutrients, conditionally essential nutrients and phytonutrients.

These terms are not necessarily mutually exclusive. For example, certain nutrients may be defined as either a macronutrient or a micronutrient depending on the particular classification system or list. The expression “at least one nutrient” or “one or more nutrients” means, for example, one, two, three, four, five, ten, 20 or more nutrients.

The term “determining a level of one or more nutrients” includes determining metabolites and/or biomarkers of individual nutrients. Thus in some embodiments, a level of e.g. a metabolite or other indicator of one or more of the above nutrients is measured.

Macronutrients

The term “macronutrient” is well known in the art and is used herein according to it standard meaning to refer to a nutrient which is required in large amounts for the normal growth and development of an organism.

Macronutrients include, but are not limited to, carbohydrates, fats, proteins, amino acids and water. Certain minerals may also be classified as macronutrients, such as calcium, chloride, or sodium.

Micronutrients

The term “micronutrient” refers to compounds having a beneficial effect on the body, e.g. to provide energy, growth or health, but which are required in only minor or trace amounts. The term includes both organic and inorganic compounds, e.g. individual amino acids, nucleotides and fatty acids; vitamins, antioxidants, minerals, trace elements, e.g. iodine, and electrolytes, e.g. sodium chloride, and salts thereof.

An illustrative list of vitamins includes, vitamins A, D, E, K, B1, B2, B6, B12, and C, retinol, retinyl acetate, retinyl palmitate, beta-carotene, cholecalcipherol, ergocalcipherol, D-alpha-tocopherol, DL-alpha-tocopherol, D-alpha-tocopheryl acetate, D-alpha-tocopheryl acid succinate, phyllochinone, thiamine hydrochloride, thiamine mononitrate, riboflavin, sodium riboflavin-5′-phospate, nicotinic acid, nicotinamide, calcium-D-pantothenate, sodium-d-pantothenate, dexpanthenol, pyridoxine hydrochloride, pyridoxine-5′-phosphate, pyridoxine dipalmitate, pteroyl-monoglutamic acid, cyancobalamin, hydroxocobalamin, D-biotin, L-ascorbic acid, sodium-L-ascorbate, calcium-L-ascorbate, potassium-L-ascorbate, and L-ascorbyl-6-palmitate.

An illustrative list of minerals includes calcium (Ca), chloride (Cl), chromium (Cr), cobalt (Co) as part of Vitamin B12, copper (Cu), iodine (I), iron (Fe), fluoride (FI), magnesium (Mg), manganese (Mn), molybdenum (Mo), phosphorus (P), potassium (K), selenium (Se), sodium (Na), sulphur (S), and zinc (Zn). An illustrative list of organic acids includes, acetic acid, citric acid, lactic acid, malic acid, choline and taurine.

An illustrative list of amino acids includes, L-alanine, L-arginine, L-cysteine, L-histidine, L-glutamine acid, L-glutamine, L-isoleucine, L-leucine, L-lysine, L-methionine, L-ornithine, phenylalanine, L-threonine, L-tryptophan, L-tyrosine, and L-valine.

An illustrative list of fatty acids includes C4:0, C6:0, C8:0, C10:0, C11:0, C12:0, C13:0, C14:0, C15:0, C16:0, C17:0, C18:0, C20:0, C21:0, C22:0, C24:0, C14:1 n-5, C15:1 n-5, C16:1 n-7, C17:1 n-7, C18:1 n-9 trans, C18:1 n-9 cis, C20:1 n-9, C22:1 n-9, C24:1 n-9, C18:2 n-6 trans, C18:2 n-6 cis, C18:3 n-6, C18:3 n-3, C20:2 n-6, C20:3 n-6, C20:3 n-3, C20:4 n-6, C22:2 n-6, C20:5 n-3 and C22:6 n-3 fatty acids. In the nomenclature CX:Y, X refers to the total number of carbon atoms in the fatty acid and Y defines the total number of double bonds in the fatty acid.

Phytonutrient

The term “phytonutrient” refers to a bioactive plant-derived compound associated with positive health effects.

An illustrative, non-exhaustive list of phytonutrients includes: terpenoids (isoprenoids) such as carotenoids, triterpenoid, monoterpenes and steroids; phenolic compounds, for example natural monophenols, polyphenols (e.g. flavonoids, isoflavonoid, flavonolignan, lignans, stilbenoids, curcuminoids, stilbenoid and hydrolysable tannin); aromatic acids (e.g. phenolic acids and hydroxycinnamic acids); capsaicin; phenylethanoids; alkylresorcinols; glucosinolates; betalains and chlorophylls.

Essential Nutrient

The term “essential nutrient” is used herein to refer to a nutrient which the subject cannot synthesize endogenously, or cannot synthesize at the level required for good health. For example an essential nutrient may be a nutrient which must be obtained from the subject's diet.

An illustrative, non-exhaustive list of essential nutrients includes essential fatty acids, essential amino acids, essential vitamins and essential dietary minerals.

Essential amino acids for humans include phenylalanine, valine, threonine, tryptophan, methionine, leucine, isoleucine, lysine and histidine.

Essential fatty acids for humans include alpha-linolenic acid and linoleic acid.

In addition, the nutrient may be “conditionally essential” depending on, for example, whether the subject has a specific disease, condition or genotype.

User of System and Methods

In several embodiments, the user is a typical consumer of food and beverage products as well as nutritional supplements.

In some embodiments, the user is a retailer who may be interested in optimizing their recommendations of food and beverage products and nutritional supplements in consideration of an individualized product offering.

In some embodiments, the user is a health care professional who is interested in recommending food and beverage products and nutritional supplements with an individualized recommended nutrient content for their client.

In some embodiments, the user is interested in food and beverage products and nutritional supplements for human use.

In other embodiments, the user is interested in food and beverage products and nutritional supplements for animal use, particularly companion animals such as dogs and cats.

Systems and Methods

The disclosed systems and methods of the invention improve upon the general population-based recommendation methods for calculating recommended daily intake of nutrients by recognizing the importance of individual genetic variation. This is determined, according to the invention, by analyzing the individual's SNPs from a DNA sample and providing a weighted polygenic risk score per nutrient and adjusting the individual recommended daily intake for at least one nutrient of a plurality of nutrients. For instance, each respective nutrient may have a different genetic risk score component which influences the overall nutrient supplement, food, diet, meal, menu, and recipe recommendations for an individual.

The provided systems and methods additionally improve methods for recommended daily intake of nutrients by collecting individual genetic data via genetic tests thus advantageously provide more accurate RDAs per individual.

In various embodiments, the system and methods disclosed herein use the full knowledge and distributional properties of the RDA or other intake recommendation regimes. In some embodiments, the disclosed system and method takes into account the upper and lower limits of intake recommendations for each nutrient. In some embodiments of the system disclosed herein use a combination of EAR and RDA for determining the reference RDA in a population and making the comparison between the reference RDA and the individual subject RDA. The disclosed system can then determine a personalized, RDA amount per nutrient for an individual over a given period of time.

In several embodiments, based on information from the food intake of the individual, it is possible to determine according to the invention whether an individual is “low”, “average” or “high” by comparing the average population RDA or reference RDA per nutrient compared to the personalized RDA. In this way, in some embodiments, the system and methods disclosed herein enable measurement of how far the actually-consumed nutrient amounts are from the recommended personalized RDAs per nutrient over the daily consumption of the individual user.

In some embodiments, the nutrient score for a particular nutrient whose intake sufficiency is defined in terms of an Adequate Intake (AI) value is calculated as the smaller of a maximum score and the percentage of the AI value consumed by the individual. Thus, the system disclosed herein can calculate scores for nutrients even where the nutrient does not have established EAR and RDA values to provide a reference RDA for a particular nutrient.

Embodiments of the disclosed system offer a variety of software and analytic tools to assess, plan, and optimize nutrient supplements, foods, beverages, meals, diets, menus and recipes on an individual basis, and take into account the recommended nutrient intake amounts for an individual and how far the actual nutrient intake is from the personalized recommendation of nutrient intake. In various embodiments, the disclosed system determines the nutritional adequacy, in terms of minimum amounts to be consumed to reach the individual's recommended daily intake per nutrient.

In some embodiments, the system and methods disclosed herein can be used by nutritionists, health-care professionals, and individual users (e.g., users of wearable devices such as smart watches or fitness trackers). In several embodiments, the system disclosed herein includes at least one processor configured to execute an algorithm to calculate the individualized RDAs for at least one of plurality of nutrients. In this embodiment, the algorithm takes into account RDAs for at least one of plurality of nutrients and the amounts recommended for intake specific to an individual and compares these to the measurement of the plurality of nutrients per nutrient within that individual's diet.

In various embodiments, the user-specific inputs to the disclosed system are programmable and configurable, and include gender, age, weight, height physical activity level, whether pregnant or lactating, and the like.

In some embodiments, the system disclosed herein is configured to evaluate adequacy of nutrient intake in terms of maximal amounts. In these embodiments, the system takes into consideration toxicity and adverse effects of consuming too much of a particular nutrient or consumable.

In an embodiment, the disclosed system includes or is connected to databases containing foods or beverage composition items and respective nutrient content. In this embodiment, the disclosed system includes a fuzzy search feature that enables a user to enter a consumed (or to-be consumed) food or beverage, and thereafter searches the database to find a closest item to the user-provided item and the respect nutrient content of the food or beverage composition item may be calculated and compared to the RDA per nutrient.

In various embodiments, the disclosed system further includes an interface (e.g., a graphical user interface) to display the amount of each nutrient available in each food or beverage composing the diet. In some embodiments, this interface enables users to modify the amount of various foods or beverages to be consumed, and correspondingly displays the nutrient amounts based on the modified amount of food or beverage to be consumed. In other embodiments, the system is configured to determine amounts of food or beverage consumed using non-user-input data, such as by scanning one or more bar codes, QR codes, or RFID tags, or by tracking items ordered from a menu or purchased at a grocery store.

In some embodiments, the disclosed system includes a recommendation feature that recommends particular foods to an individual that will maximize the overall nutrient balance of the individual. In such embodiments, an algorithm executed by the disclosed system generates a list of recommendations to improve the nutrient balance to most closely optimize the recommended daily allowance of nutrients and can be a valuable tool for nutritionists for the planning and assessment of a diet.

In various embodiments, the system disclosed herein calculates one or more nutrient scores tailored to an individual based on the individual's caloric intake range and corresponding healthy ranges of nutrient intakes for a given time period. The calculated scores are based on whether nutrient intake falls within a healthy range, and are affected not only by under-consumption of nutrients but also by over-consumption of nutrients. These scores enable individuals to determine whether they are consuming enough nutrients, and to the extent they are not, to determine which additional nutrients need to be consumed. The disclosed system also makes suggestions for adding or removing consumables that, if consumed (or removed from a diet), will provide the individual with nutrients in amounts determined to be within healthy nutrient ranges for that individual.

Various embodiments of the disclosed system display a dashboard or other appropriate user interface to a user that is customized based on the user's nutritional needs, related to the individuals' RDA values per nutrient as determined by methods of the invention.

Various embodiments of the disclosed system also provide an advisory functionality. In these embodiments, after calculating the nutrient intake amounts for a first meal, the disclosed system suggests combinations of consumables that can be consumed for the remainder of the time period to result in the individual obtaining the nutrients he or she requires. For example, if an individual indicates that he or she has eaten certain foods for breakfast and lunch, the disclosed system can suggest a dinner menu that will ensure the individual gets all the nutrients he or she needs in the day while still consuming an amount of calories that falls within a caloric intake range applicable to the individual to reach the optimal RDAs per nutrient for that individual. In this embodiment, the recommendations provided by the disclosed system are optimized; the system determines the impact on the overall nutritional health score of a plurality of foods stored in its database, and suggests foods that result in an optimal increase for a plurality of nutrients.

In various embodiments, the disclosed system stores some or all of the values needed to calculate the RDAs in one or more databases. In addition, the disclosed system may store a table of caloric intake ranges for individuals based on the age, gender, and weight or Body Mass Index (BMI) of the individuals.

In another embodiment, the disclosed system provides for further customization by enabling the user to specify additional information, such as body type, physical activity level, and the like. In this embodiment, the disclosed system uses these additional inputs to adjust not only optimal caloric intake ranges for different individuals, but also for RDAs for nutrients tracked by the system. For example, if an individual indicates that he or she is athletic with a relatively high amount of athletic activity, the system may adjust the carbohydrate nutrient range upward to account for the individual's need for additional carbohydrates.

Accordingly, various embodiments of the disclosed system advantageously enable the calculation of a nutritional health score for an individual by performing the following steps:

(1) Storing reference RDAs of a plurality of nutrients, based on the recommendations of nutritional authorities

(2) Calculating and storing the individual user RDAs based on genetic information for a plurality of nutrients

(3) Storing indications of endpoints for nutrient consumption to enable the system to adjust for over- and under-consumption beyond the endpoints, as is appropriate for each nutrient

(4) Storing score weighting and individual RDAs for each nutrient and individual

(5) For a particular consumable, computing the nutrient component per nutrient and comparing it to the individuals' RDAs for that particular nutrient

(6) Providing a recommendation for the consumable by applying algorithm for each nutrient to personalize the nutrient for the individual

Various embodiments of the disclosed system further advantageously provide nutritional advice to users based on calculated nutrients. For example, embodiments of the disclosed system determine amounts of nutrients that would be needed to place an individual in the healthy amount range for those nutrients. These embodiments then analyze a database of the RDAS per consumables (e.g., foods, beverages or ingredients) to determine combinations of consumables that will provide the needed amounts of nutrients to place the user in the healthy amount ranges while still remaining within the optimal caloric intake range for that individual taking into account the genetic information of the individual in terms of the RDAs per nutrient.

In various embodiments, the disclosed system works in conjunction with a laboratory or other testing facility that generates actual data about individuals using the disclosed system. For example, in one embodiment the disclosed system enables a user to submit to a DNA test to determine the SNP profile of the individual and to calculate the individual's RDA for a plurality of nutrients.

In another embodiment, the disclosed system enables a user to submit to a further test, such as a blood spot test, to determine whether the individual is over- or under-consuming various nutrients. In such embodiments, this testing and lab work enables the system to verify that its recommendations are working, that is, to verify that a user is actually receiving adequate nutrients when the scoring function indicates that his or her intake ranges are within the desired range. In various embodiments, other bodily fluids (e.g., urine, saliva, etc.) can be used to perform these verifications. In these embodiments, the data of the user's actual bodily fluid makeup can allow the system to calibrate itself to ensure that an overall nutrient score can be calculated for an individual actually means that the individual is receiving adequate amounts of that nutrient. For example, to determine whether a score of 100 for a particular RDA of a nutrient is met, the system may use fluid measurements to determine whether the individual actually is receiving sufficient amounts of that nutrient. In the event the individual is receiving too little (or too much) of a given nutrient, the fluid measurement results can be used to alter the scoring algorithms to ensure that a score of 100 actually reflects an ideal intake of a particular nutrient for a particular individual.

In various embodiments, one or more of the inputs referenced above is pulled from a database of nutritional information. For example, the list of consumed nutrients may be generated in certain embodiments by allowing a user to enter a consumed item and looking up a listing of nutrients contained in that consumed item in an appropriate consumables database. In other embodiments, users enter consumed nutrients directly. In still other embodiments, a user enters a food (e.g., a hamburger) and if that food is not within a database, the user also enters an amount of nutrients within that food (e.g., an amount of sodium). Thereafter, future entries of the defined food (e.g., a hamburger) can lookup nutrients entered at a previous time rather than requiring the user to re-enter the nutrient information.

In some embodiments, the disclosed system includes a functionality to use several measure units and serving sizes, specific to a particular food, and automatically convert between them. Therefore, a portion of one food can be entered as a given amount of grams, of kilo-calories, or according to some pre-defined serving size (e.g. cup, tablespoon, etc.), and can be converted (or normalized) to an amount of food consumed that is compatible with data stored in the database about the food.

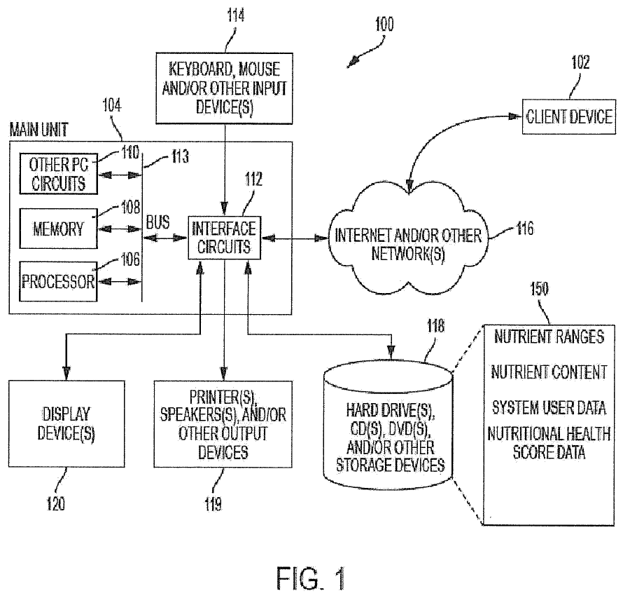

Referring now to FIG. 1, a block diagram is illustrated showing an example of the electrical systems of a host device 100 usable to implement at least portions of the computerized recommendation system and the recommended intakes of nutrients disclosed herein.

In one embodiment, the device 100 illustrated in FIG. 1 corresponds to one or more servers and/or other computing devices that provide some or all of the following functions: (a) enabling access to the disclosed system by remote users of the system; (b) serving web page(s) that enable remote users to interface with the disclosed system; (c) storing and/or calculating underlying data, such as recommended caloric intake ranges, recommended personalized nutrient consumption ranges, and nutrient content of foods, needed to implement the disclosed system; (d) calculating and displaying component recommended daily intakes of nutrients or aggregate nutritional health scores; and/or (e) making recommendations of foods or other consumables that can be consumed to help individuals reach optimal recommended daily intakes or nutritional health scores.

In the example architecture illustrated in FIG. 1, the device 100 includes a main unit 104 which preferably includes one or more processors 106 electrically coupled by an address/data bus 113 to one or more memory devices 108, other computer circuitry 110, and/or one or more interface circuits 112. The one or more processors 106 may be any suitable processor, such as a microprocessor from the INTEL PENTIUM® or INTEL CELERON® family of microprocessors. PENTIUM® and CELERON® are trademarks registered to Intel Corporation and refer to commercially available microprocessors. It should be appreciated that in other embodiments, other commercially-available or specially-designed microprocessors may be used as processor 106. In one embodiment, processor 106 is a system on a chip (“SOC”) designed specifically for use in the disclosed system.

In one embodiment, device 100 further includes memory 108. Memory 108 preferably includes volatile memory and non-volatile memory. Preferably, the memory 108 stores one or more software programs that interact with the hardware of the host device 100 and with the other devices in the system as described below. In addition or alternatively, the programs stored in memory 108 may interact with one or more client devices such as client device 102, discussed in detail below, to provide those devices with access to media content stored on the device 100. The programs stored in memory 108 may be executed by the processor 106 in any suitable manner.

The interface circuit(s) 112 may be implemented using any suitable interface standard, such as an Ethernet interface and/or a Universal Serial Bus (USB) interface. One or more input devices 114 may be connected to the interface circuit 112 for entering data and commands into the main unit 104. For example, the input device 114 may be a keyboard, mouse, touch screen, track pad, track ball, isopoint, and/or a voice recognition system. In one embodiment, wherein the device 100 is designed to be operated or interacted with only via remote devices, the device 100 may not include input devices 114. In other embodiments, input devices 114 include one or more storage devices, such as one or more flash drives, hard disk drives, solid state drives, cloud storage, or other storage devices or solutions, which provide data input to the host device 100.

One or more storage devices 118 may also be connected to the main unit 104 via the interface circuit 112. For example, a hard drive, CD drive, DVD drive, flash drive, and/or other storage devices may be connected to the main unit 104. The storage devices 118 may store any type of data used by the device 100, including data regarding preferred nutrient ranges, data regarding nutrient contents of various food items, data regarding users of the system, data regarding previously-generated individual recommended daily intakes of nutrients, data regarding nutritional health scores, data representing weighting values for calculating nutritional health scores, sensitivity values for calculating nutritional health scores, and any other appropriate data needed to implement the disclosed system, as indicated by block 150. Alternatively or in addition, storage devices 118 may be implemented as cloud-based storage, such that access to the storage 118 occurs via an internet or other network connectivity circuit such as an Ethernet circuit 112.

One or more displays 120, and/or printers, speakers, or other output devices 119 may also be connected to the main unit 104 via the interface circuit 112. The display 120 may be a liquid crystal display (LCD), a suitable projector, or any other suitable type of display. The display 120 generates visual representations of various data and functions of the host device 100 during operation of the host device 100. For example, the display 120 may be used to display information about the database of preferred nutrient ranges, a database of nutrient contents of various food items, a database of users of the system, a database of previously-generated individual recommended daily intakes of nutrients, a databased or nutritional health scores, and/or databases to enable an administrator at the device 100 to interact with the other databases described above.

In the illustrated embodiment, the users of the computerized personalized nutrient recommendation system interact with the device 100 using a suitable client device, such as client device 102. The client device 102 in various embodiments is any device that can access content provided or served by the host device 100. For example, the client device 102 may be any device that can run a suitable web browser to access a web-based interface to the host device 100. Alternatively or in addition, one or more applications or portions of applications that provide some of the functionality described herein may operate on the client device 102, in which case the client device 102 is required to interface with the host device 100 merely to access data stored in the host device 100, such as data regarding individual daily recommendations of daily intakes of nutrient ranges or nutrient content of various food items.

In one embodiment, this connection of devices (i.e., the device 100 and the client device 102) is facilitated by a network connection over the Internet and/or other networks, illustrated in FIG. 1 by cloud 116. The network connection may be any suitable network connection, such as an Ethernet connection, a digital subscriber line (DSL), a Wi-Fi connection, a cellular data network connection, a telephone line-based connection, a connection over coaxial cable, or another suitable network connection.

In one embodiment, host device 100 is a device that provides cloud-based services, such as cloud-based authentication and access control, storage, streaming, and feedback provision. In this embodiment, the specific hardware details of host device 100 are not important to the implementer of the disclosed system-instead, in such an embodiment, the implementer of the disclosed system utilizes one or more Application Programmer Interfaces (APIs) to interact with host device 100 in a convenient way, such as to enter information about the user's demographics to help determine healthy nutritional ranges, to enter information about consumed foods, and other interactions described in more detail below.

Access to device 100 and/or client device 102 may be controlled by appropriate security software or security measures. An individual user's access can be defined by the device 100 and limited to certain data and/or actions, such as inputting consumed food or viewing calculated scores, according to the individual's identity. Other users of either host device 100 or client device 102 may be allowed to alter other data, such as weighting, sensitivity, or healthy range values, depending on those users' identities. Accordingly, users of the system may be required to register with the device 100 before accessing the content provided by the disclosed system.

In a preferred embodiment, each client device 102 has a similar structural or architectural makeup to that described above with respect to the device 100. That is, each client device 102 in one embodiment includes a display device, at least one input device, at least one memory device, at least one storage device, at least one processor, and at least one network interface device. It should be appreciated that by including such components, which are common to well-known desktop, laptop, or mobile computer systems (including smart phones, tablet computers, and the like), client device 102 facilitates interaction among and between each other by users of the respective systems.

In various embodiments, devices 100 and/or 102 as illustrated in FIG. 1 may in fact be implemented as a plurality of different devices. For example, the device 100 may in actuality be implemented as a plurality of server devices operating together to implement the media content access system described herein. In various embodiments, one or more additional devices, not shown in FIG. 1, interact with the device 100 to enable or facilitate access to the system disclosed herein. For example, in one embodiment the host device 100 communicates via network 116 with one or more public, private, or proprietary repositories of information, such as public, private, or proprietary repositories of nutritional information, nutrient content information, healthy range information, environmental impact information, or the like.

In one embodiment, the disclosed system does not include a client device 102. In this embodiment, the functionality described herein is provided on host device 100, and the user of the system interacts directly with host device 100 using input devices 114, display device 120, and output devices 119. In this embodiment, the host device 100 provides some or all of the functionality described herein as being user-facing functionality.

The system of FIG. 1 is configured to calculate the individual user's recommended daily intakes per nutrient based on genetic information and overall nutrient scores based on the foods consumed or to be consumed during the day. Those of skill in the art will understand that this functionality is not general-purpose computer functionality, but requires the computer to be specially programmed with instructions to calculate results the recommended daily intake per nutrient for a specific individual user based on genetic information performed using the various algorithms described in various embodiments herein.

In various embodiments, the system disclosed herein is arranged as a plurality of modules, wherein each module performs a particular function or set of functions. The modules in these embodiments could be software modules executed by a general purpose processor, software modules executed by a special purpose processor, firmware modules executing on an appropriate, special-purpose hardware device, or hardware modules (such as application specific integrated circuits (“ASICs”)) that perform the functions recited herein entirely with circuitry. In embodiments where specialized hardware is used to perform some or all of the functionality described herein, the disclosed system may use one or more registers or other data input pins to control settings or adjust the functionality of such specialized hardware. For example, a hardware module may be used that is programmed to analyze the plurality of recommended daily intakes of nutrients per nutrient. In still other embodiments, where the modules to perform various functionality described herein are software modules executable by hardware, the modules may take the form of apps or subsets of apps that may be designed to run on a processor executing a particular, predefined operating system environment.

In another embodiment, one or more devices carried by the user provide real-time information to the system when the user is in a food purchasing establishment such as a grocery store or a restaurant. Devices such as RFID readers, NFC readers, wearable camera devices, and mobile phones could receive or determine (such as by scanning RFID tags, reading bar codes, or determining the physical location of a user) foods that are available to a user at a particular grocery store or restaurant. The disclosed system then makes recommendations taking into account what foods could be immediately purchased or consumed by the user. In one such embodiment, when a user sits down at a restaurant, the disclosed system may push information to the user's mobile phone recommending that the user select certain items from the menu to optimize the user's nutritional health score for a given time period. In still other embodiments, a voice recognition feature recognizes inputs provided vocally by a user. In one such embodiment, the voice recognition system listens as a user orders at a restaurant; in other embodiments, the voice recognition system enables the user to speak directly the items he or she has consumed or will consume.

In one embodiment, the system disclosed herein calculates a score between 0.0 and 100 for each nutrient. In this embodiment, the intake range (and thus the scoring function) are divided into three different regions: (1) intakes of the nutrient between 0.0 and the lower consumption limit, which may be based on a combination of the EAR and RDA; (2) intakes between the lower consumption limit and the upper limit, which is equivalent to UL; and (3) intakes above the upper consumption limit. In this embodiment, intakes in the first range indicate a potential deficiency; the nearer the intake amount is to the RDA, the less likely the intake is actually insufficient. The second range may be described as the “hemostasis region,” wherein the score approaches and/or is equal to the maximum value for that nutrient (e.g., 100). The third range reflects over-consumption, and is an intake range whereby chronic intakes in the range are generally not recommended. In the described embodiment, scores for a particular nutrient within the third range decrease until they reach a minimum score (e.g., 0).

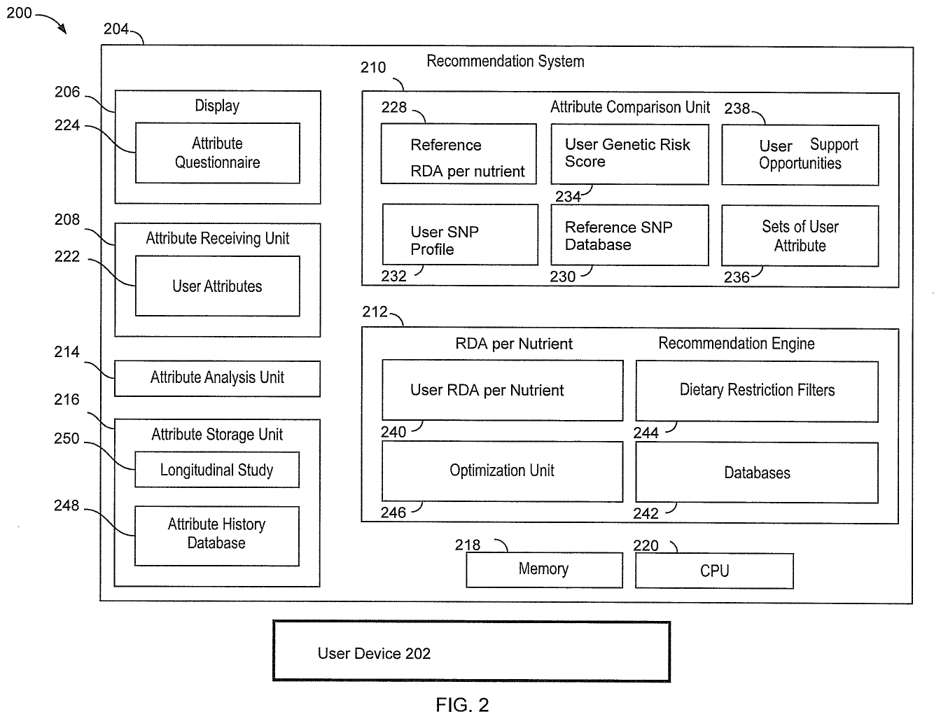

FIG. 2 illustrates an example system according to an embodiment of the present disclosure. The system 200 includes a user device 202 and a recommendation system 204. In another embodiment of the present disclosure, the recommendation system 204 can be incorporated as an example of the embodiment of the recommendation system 150 of FIG. 1. The user device 202 may be implemented as a computing device, such as a computer, smartphone, tablet, smartwatch, or other wearable through which an associated user can communicate with the recommendation system 204. The user device 202 may also be implemented as, e.g., a voice assistant configured to receive voice requests from a user and to process the requests either locally on a computer device proximate to the user or on a remote computing device (e.g., at a remote computing server).

In another embodiment, the user device 202 may be a dispensing device which communicates with the recommendation system 204 to receive nutrient recommendations for the user and then dispenses nutrient supplements, foods, beverages, meals, menus or recipes which have been personalized to the individual user of the device based on the user's recommended daily intake data from the recommendation engine 212.

The recommendation system 204 includes one or more of a display 206, an attribute receiving unit 208, an attribute comparison unit 210, an evidence-based diet and lifestyle recommendation engine 212, an attribute analysis unit 214, an attribute storing unit 216, a memory 218, and a CPU 220. Note, that in some embodiments, a display 206 may additionally or alternatively be located within the user device 202. In an example, the recommendation system 204 may be configured to receive a request for a plurality of individual RDAs per nutrient 240. For example, a user may install an application on the user device 202 that requires the user to sign up for a recommendation service. By signing up for the service, the user device 202 may send a request for the personalized RDAs per nutrient 240. In a different example, the user may use the user device 202 to access a web portal using user-specific credentials. Through this web portal, the user may cause the user device 202 to request personalized RDA recommendations from the recommendation system 204.

In another example, the recommendation system 204 may be configured to request and receive a plurality of user attributes 222. For example, the display 206 may be configured to present an attribute questionnaire 224 to the user. The attribute receiving unit 208 may be configured to receive the user attributes 222. In one example, the attribute receiving unit 208 may receive a plurality of answers 226 based on the attribute questionnaire 224, and based on the plurality of answers, determine the plurality of user attributes 222. For example, the attribute receiving unit 208 may receive answers to the attribute questionnaire 224 suggesting that the diet of the user is equivalent to the recommended dietary allowance (“RDA”) and then determine the user attributes 222 to be equivalent to the RDA, of Vitamin D per day. In another example, the user device attribute receiving unit 208 may directly receive the user attributes 222 from the user device 202.

In another example, the attribute receiving unit 208 may be configured to receive the test results of a DNA test kit, the results of a standardized health test administered by a medical professional, the results of a self-assessment tool used by the user, or the results of any external or third party test. Based on the results from any of these tests or tools, the attribute receiving unit 208 may be configured to determine the user attributes 222. For example, the SNP profile of the user may be determined from the DNA testing kit before the intervention of the RDA nutrient recommendations. The individual user polygenic risk score can be calculated per nutrient to determine the RDA nutrient recommendations per nutrient for the individual user. The same measurements may be determined at a time period after the new personalized RDA interventions per nutrient to determine whether there has been an improvement or maintenance of the health status of the user.

The attribute comparison unit 210 may be configured to determine a user genetic risk score 234 based on the comparison between the reference RDA per nutrient benchmarks 228 and the user attributes 222. For example, a score may be represented through lettering grades, symbols, or any other system of ranking, for example, “high”, “average”, “low” or “above average”, “average”, “below average”, that allows a user to interpret how well their current attributes rate amongst benchmark references and where they may have a nutrient deficiency based on their genetic risk profile for a particular nutrient.

The recommendation system 204 may be further configured to determine a plurality of support opportunities 238 based on the plurality of user attributes 222. The recommendation system 204 may be further configured to identify a plurality of healthy recommendations 240 based on the plurality of support opportunities 238. For example, the evidence-based diet and lifestyle recommendation engine 212 may be configured to be cloud-based. The recommendation engine 212 may comprise one or more of a plurality of databases 242, a plurality of dietary restriction filters 244, and an optimization unit 246. Based on the plurality of opportunities 238, the recommendation engine 212 may identify the plurality of healthy recommendations 240 according to the one or more of plurality of databases 242, the dietary restriction filters 244, and the optimization unit 246.

In one example, the recommendation engine 212 may connected to further databases 242 such a food database, beverage database, nutrient supplement database, menu database, recipe database, diet database, all of which are annotated by nutrient composition per nutrient.

In another example, the recommendation engine 212 may connected to the dietary restriction filter 244 which may note any user food allergies or personal food or beverage preferences.

In another example, the recommendation system 204 may be configured to provide continuous recommendations, based on prior user attributes. For example, the recommendation system 204 may comprise, in addition to the previously discussed elements, an attribute storing unit 216 and an attribute analysis unit 214. The attribute storing unit 216 may be configured to, responsive to the attribute receiving unit 108 receiving the plurality of user attributes 222, add the received user attributes 222 to an attribute history database 248 as a new entry based on when the plurality of user attributes 222 were received. For example, if user attributes 222 are received by the attribute receiving unit 208 on a first meal of the day, the attribute storing unit 216 will add the received user attributes 222 to a cumulative attribute history database 248 noting the date of entry, in this case the first meal of the day. Later, if user attributes 222 are received by the attribute receiving unit 208 on a second meal or further meal of the day, the attribute storing unit 216 will also add these new attributes to the attribute history database 248, noting that they were received on the second meal or further meal of the day, while also preserving the earlier attributes from the first meal of the day in order to calculate the total nutrient content per nutrient in order to reach the personalized recommended daily intake per nutrient per day.

This attribute analysis unit 214 may be configured to analyze the plurality of user attributes 222 stored within the attribute history database 248, wherein analyzing the stored plurality of user attributes 222 comprises performing a longitudinal study 250. Continuing the earlier example, the attribute analysis unit 214 may perform a longitudinal study of the user attributes 222 from each of the first day, the second day, and every other collection of user attributes 222 found within the attribute history database 248. The evidence based diet and lifestyle recommendation engine 212 may be further configured to generate a plurality of healthy recommendations 240 based on at least the stored user attributes 222 found within the attribute history database 248 and the analysis performed by the attribute analysis unit 214.

In an embodiment, the attribute analysis unit 214 is further configured to repeatedly analyze the plurality of user attributes 222 stored within the attribute history database 248 responsive to the attribute storing unit 216 adding a new entry to the attribute history database 248, essentially re-analyzing all of the data within the attribute history database 248 immediately after new user attributes 222 are received. Similarly, the evidence based diet and lifestyle recommendation engine 212 may be further configured to repeatedly generate the plurality of healthy recommendations 240 responsive to the attribute analysis unit 214 completing an analysis, thereby effectively generating new healthy recommendations 240 that consider all past and present user attributes 222 each time a new set of user attributes 222 is received.

In various embodiments, the user-specific inputs to the disclosed system are programmable and configurable, and include gender, age, weight, height, physical activity level, BMI, and the like.

In an embodiment, the disclosed system includes or is connected to a plurality of databases 242 containing foods or beverage items, meals, menus or recipes and their respective nutrient content per nutrient. In this embodiment, the disclosed system includes a fuzzy search feature that enables a user to enter a consumed (or to-be consumed) food or beverage, and thereafter searches the database to find a closest item to the user-provided item. The disclosed system, in this embodiment, uses stored nutritional information about the matched food item to determine whether it is a healthy item with respect to the RDAs per nutrient and whether it is a good choice based on the total RDA required per nutrient per day.

In various embodiments, the disclosed system further includes an interface (e.g., a graphical user interface) to display the amount of each nutrient available in each food composing the diet, and displays the amount of energy available to be consumed. In some embodiments, this interface enables users to modify the amount of various foods or energy to be consumed. In other embodiments, the system is configured to determine amounts of food or energy consumed using non-user-input data, such as by scanning one or more bar codes, QR codes, or RFID tags, image recognition systems, or by tracking items ordered from a menu or purchased at a grocery store.

Various embodiments of the disclosed system display a dashboard or other appropriate user interface to a user that is customized based on the user's needs. In embodiments of the system disclosed herein, a graphical user interface is provided which advantageously enables, for the first time, users to input data about food consumed in a given period of time and to see an indication of a score, based appropriately on energy consumption, that reflects overall nutritional content of the consumed diet.

In several embodiments, the present invention provides methods for recommending daily intake of nutrients based on the individual genetic information thus providing more accurate nutritional recommendations.

In several embodiments, the present invention provides methods of improving or maintaining nutritional health by providing food, beverage or nutritional supplement recommendations based on individual genetic information.

In several embodiments, the present invention provides methods of improving or maintaining nutritional health by providing by meal, menu or recipe recommendations based on individual genetic information.

In one embodiment of the invention, the method comprises determining recommended daily intake of nutrients for an individual wherein said method comprises the steps of:

(i) determining the SNP genotype profile of an individual subject from a DNA sample from said subject;

(ii) comparing the SNP genotype profile of an individual subject to a reference SNP genotype profile;

(iii) calculating the individual's genetic risk score per nutrient for at least one of a plurality of nutrients;

(iv) comparing the reference recommended daily intake of said nutrient to the new recommended daily intake of said nutrient based on the individual's genetic risk score per nutrient for at least one of a plurality of nutrients; and

(v) providing the new recommended daily intake to the individual for at least one of a plurality of nutrients.

In another embodiment of the invention, the method provides the personalised recommended daily intake based on the individual's genetic risk score per nutrient which is classified as high, normal or low compared to the reference recommended daily intake per nutrient for at least one of a plurality of nutrients.

In a preferred embodiment of the invention, the method of the invention provides the SNP genotype profile of an individual subject determined from a DNA sample from said subject by buccal swab.

In another embodiment of the invention, the method provides the personalised recommended daily intake based on the individual's genetic risk score per nutrient wherein the at least one of a plurality of nutrients are selected from the group comprising vitamins and/or minerals.

In a preferred embodiment, the at least one of a plurality of nutrients is Vitamin D.

In another embodiment of the invention, the method provides the personalised recommended daily intake based on the individual's genetic risk score per nutrient wherein the new recommended daily intake for at least one of a plurality of nutrients recommends nutrient supplements to meet the new recommended daily intake of at least one of a plurality of nutrients.

In another embodiment of the invention, the method provides the personalised recommended daily intake based on the individual's genetic risk score per nutrient wherein the new recommended daily intake for at least one of a plurality of nutrients recommends foods, beverages and/or daily meal planning to meet the new recommended daily intake of at least one of a plurality of nutrients.

In a preferred embodiment of the invention, the method is a computer-implemented invention.

In an embodiment of the invention, the computer implemented method provides the personalised recommended daily intake based on the individual's genetic risk score per nutrient wherein the new recommended daily intake for at least one of a plurality of nutrients comprises:

(i) collecting individual user daily consumable nutrient intake data via interaction with a computer user interface;

(ii) calculating the individual user daily consumable nutrient intake data per consumable event at different time points during the day; and

(iii) providing recommendations via a computer user interface for the recommended daily allowance per nutrient for the individual user by recommending nutrient supplements, foods, beverages and/or meals for the remaining time during the day to meet the new recommended daily intake for that individual user for at least one of a plurality of nutrients.

In another embodiment of the invention, the computer implemented method provides the personalised recommended daily intake based on the individual's genetic risk score per nutrient wherein said individual user's recommended daily intake for nutrients is connected to a dispensing device which dispenses nutrient supplements, foods, beverages or complete meals with the recommended daily intake of at least one of a plurality of nutrients that have been personalized for that individual user.

In several embodiments, the recommendations for nutrient supplements, foods, beverages, meals, diets, menus or recipes take into account the general nutritional composition and overall daily energy consumption recommended for the individual user.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of and “consisting of the disclosed components. Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

### EXAMPLES

### Example 1: Genetic Prediction of Vitamin D Level for an Individual

From a list of candidate SNPs associated with vitamin D level in European populations, a genetic score model was designed for prediction. SNPs associated with vitamin D level in genome-wide association study (GWAS) literature were extracted from the GWAS catalog (https://www.ebi.ac.uk/gwas/), a curated database holding publicly available results fromlarge-scale GWASs. To filter this database, the focus was on results from GWAS performed in populations of European (Caucasian origin) testing the association to ‘vitamin D measurement’ (not ‘vitamin D deficiency’) as primary endpoint. In particular, two GWAS references were informative: Ahn et al (doi:10.1093/hmg/ddq155) GWAS based on 4501 subjects; and Manousaki et al (doi:10.1016/j.ajhg.2017.06.014) GWAS based on 42,274 subjects. A set of 19 SNPs with corresponding effect size in nmol/l was extracted.

To avoid association signal redundancy, a pruning to filter SNPs was applied starting from the SNP with the lower p-value (strongest association signal) and excluding all subsequent SNPs with a r2>0.25 as proxy (i.e. carrying the same association signal). The same process was repeated for each subsequent SNP not filtered out because in weak linkage equilibrium with the previous one. The European 1000 Genome reference population was used as reference population to compute LD using PLINK tool (https://www.cog-genomics.org/plink2/).

Method

Vitamin D level associated to different genetic combinations (or “scores”) was computed theoretically.

Assuming 16 informative bi-allelic genetic markers, all combination were computed (3\hat{ }16=43,046,721 different scores). All scores were not observed in the Danish population because of their very low frequency and population genetic selection in Scandinavian. There was an assumption of a strong independence between the genetic variants (no interaction). For each score, a value of unit increase was computed as the sum over the different genotypes of the number of risk allele multiplied by the corresponding unit increase available in GWAS catalog (Table 1 below).

Score reference was defined as the combination of homozygote genotypes for the non-risk allele (i.e. 0 risk allele count). Vitamin D prevalence in Denmark was set to 40.17 nmol/l as computed from Hansen et al. (2008; doi:10.3390/nu10111801) using a weighted (population size) computation of vitamin D levels in non-supplement users men (n=1048) and women (n=1517; see Table 1 of Hansen et al. The large (5%-95%) intervals associated to values available in the table of Hansen et al. was noted.

Using this mean value and the mean of the score-associated unit increased distribution, the vitamin D associated value for the reference score was deduced. For each score, the vitamin D level was then computed as the vitamin D reference score value plus the score associated unit increase.

Results

Table 1 summarizes information from selected candidate SNPs after pruning.

TABLE 1
SNPs risk allele with associated effect size and frequency from GWAS catalog

| SNP | Chr | Bp | Risk•Allele | RAF | Effect•Size | Mapped gene |
|---|---|---|---|---|---|---|
| rs2282679 | 4 | 71742666 | T | 0.74 | 0.38 | GC |
| rs3829251 | 11 | 71483513 | G | 0.81 | 0.18 | NADSYN1 |
| rs2060793 | 11 | 14893764 | A | 0.41 | 0.25 | CALCB, CYP2R1 |
| rs55665837 | 11 | 14473503 | C | 0.627 | 0.08 | COPB1 |
| rs116970203 | 11 | 14855172 | G | 0.975 | 0.43 | PDE3B |
| rs4423214 | 11 | 71462208 | T | 0.697 | 0.1 | NADSYN1 |
| rs3819817 | 12 | 95984993 | T | 0.21 | 0.04 | HAL |
| rs2277458 | 14 | 39114277 | G | 0.214 | 0.05 | GEMIN2 |
| rs148189294 | 4 | 71575200 | G | 0.962 | 0.22 | SLC4A4, GC |
| rs4588 | 4 | 71752606 | G | 0.717 | 0.25 | GC |
| rs1607741 | 4 | 71853316 | C | 0.684 | 0.12 | AC068721.1, NPFFR2 |
| rs79761689 | 4 | 72005565 | T | 0.951 | 0.19 | NPFFR2, AC068721.1 |
| rs138485827 | 4 | 72166226 | C | 0.928 | 0.13 | NPFFR2, ADAMTS3 |
| rs78862524 | 4 | 72305473 | C | 0.947 | 0.16 | ADAMTS3 |
| rs185378533 | 11 | 71422087 | G | 0.461 | 0.07 | AP002387.1 |
| rs6127099 | 20 | 54114863 | A | 0.712 | 0.06 | CYP24A1, BCAS1 |

Score Specific Vitamin D Level

Theoretical Computation

Mean score associated unit value was estimated to 2.71 nmol/l increase when compared to the reference score. Assuming a 40.17 nmol/l vitamin D prevalence, vitamin D for the reference score was estimated to 37.46 nmol/l. For each score ranging from 1 to 32, the vitamin D level was computed as the vitamin D reference score value plus the score associated unit increase. For example, a subject homozygous carrier for each of the 16 loci, the unit increase was estimated to 5.42 and the vitamin D level to 37.46+5.42=42.88 nmol/L. Vitamin D conditional on score took values from 37.46 to 42.88 nm/I.

Application to 1000 Genome Population

The score computation process was then applied to the European 1000 Genome population (n=379 subjects). This population is expected to be representative at the genetic level of a European population. Genetic information was extracted for the 16 SNPs and scores computed. A unit increase mean was estimated to be 4.297 nmol/l; twice the 2.71 nmmol/l value computed theoretically. The number of unique score was 360 (in 379 subjects) for 43,046,721 possible combinations. The scores ranged from 16 to 31 and vitamin D conditional on score took values from 38.36 to 41.25 mol/l (assuming a 4.297 nmol/l mean unit increase). Using the 2.71 nmmol/l theoretical value, vitamin D ranged from 39.95 nmol/l and 42.84 nmol/l.

This population was not representative of the general population of score for vitamin D may be likely due to the ‘founder population’ characteristics and sample size. When considering cumulative score frequencies of all genotypic combinations, counts between 18 and 27 risk allele (within the range observed for the 1000 Genome cohort) were observed as the most frequent counts.

An appropriate cut-off for determining adequate 25(OH)D concentrations was 50 nmol/L, as this was the consensus in Denmark (Danish Health Authority. Recommendations for Vitamin D (in Danish. https://www.sst.dk/da/sundhed-og-livsstiVernaering/d-vitamin (accessed on 23 Oct. 2018)). As such, 25(OH)D concentrations below 25 nmol/L were deemed to indicate vitamin D “deficiency”, whereas concentrations between 25-50 nmol/L indicate “insufficiency”. Assuming 40.17 nmol/l prevalence in a non-supplemented Danish population during spring and the current computation process, it was expected theoretically that all tested individuals would present vitamin D insufficiency. Changing prevalence to higher values as observed in Hansen et al. for non-supplement user would likely modify theoretical vitamin D level to higher values (50+ women for instance)

As noted previously, because of different effect size between SNPs, for a given genetic score, different associated risks and then vitamin D levels are associated. The number of risk alleles as “unit of measure” was used to compute vitamin D mean levels as provided in Table 2. Genetic score was grouped in classes in Table 3.

TABLE 2
Mean vitamin D level per genetic score
defined as number of risk allele count

| GS Risk Allele | | | |
|---|---|---|---|
| Count | Mean Vitamin D | 95% CI inf | 95% CI sup |
| 16 | 38 | 37.95 | 38.84 |
| 17 | 39 | 38.55 | 39.37 |
| 18 | 39 | 38.78 | 39.29 |
| 19 | 39 | 38.97 | 39.47 |
| 20 | 40 | 39.38 | 39.63 |
| 21 | 40 | 39.57 | 39.78 |
| 22 | 40 | 39.75 | 39.92 |
| 23 | 40 | 39.92 | 40.10 |
| 24 | 40 | 40.13 | 40.28 |
| 25 | 40 | 40.29 | 40.45 |
| 26 | 40 | 40.39 | 40.53 |
| 27 | 41 | 40.64 | 40.76 |
| 28 | 41 | 40.79 | 40.92 |
| 29 | 41 | 40.92 | 41.04 |
| 30 | 41 | 41.00 | 41.23 |
| 31 | 41 | 41.19 | 41.28 |

TABLE 3
Mean vitamin D level per group of genetic scores

| GS - risk allele count | Mean vitamin D | 95% CI inf | 95% CI sup |
|---|---|---|---|
| (15, 19] | 39 | 38.89 | 39.2 |
| (19, 23] | 40 | 39.73 | 39.85 |
| (23, 27] | 40 | 40.38 | 40.46 |
| (27, 31] | 41 | 40.92 | 41.03 |

Improving the Model

Large variability in vitamin D were observed in the Danish population (Hansen et al. 2018; doi:10.3390/nu10111801). Main factors include the gender and the age as well as the season and supplementation of vitamin D. Such co-factors need to be taken into consideration when predicting vitamin D level based on genetic information from a reference sample population. Here a stable vitamin D prevalence was set to 40.17 nmol/l but the model could also be adapted to different vitamin D values based on age, gender, season and vitamin D supplementation information from the tested subject.

Vitamin D Assessment Tool Script

A script was developed in the program R to compute vitamin D level using parameters based on the current example. It can be adapted to different models of nutrients depending on differences in parameters such as different set number of SNPs, different SNP unit increase values and then different score mean unit increase for different nutrients.

### Example 2—New Recommended Daily Intake of Nutrients

Based on the age, gender, BMI and ethnicity, the reference value for Subject 1 for vitamin D levels was 37.46 nmol/L. The new recommended daily intake for Vitamin D for Subject 1 was calculated after taking a DNA sample via buccal swab and analyzing the SNP profile of Subject 1. Subject 1 was a homozygous carrier of 16 loci for Vitamin D which meant that based on the subject's genetic risk score the personalized new recommended daily intake of Vitamin D would need to be altered.

For Subject 1, the recommended daily intake based on the genetic risk score was 42.88 nmol/L. Subject 1 would need to increase the Vitamin D intake by 5.42 nmol/L in order to reach a total amount of 42.88 nmol/L. This new recommendation to increase the RDA of Vitamin D intake could be reached by a recommendation of a daily nutritional supplement of Vitamin D of 5.42 nmol/L. The recommendation could also be reached by recommendation of specific foods, beverages, meals, menus and recipes that would allow this total daily recommendation for Vitamin D to be reached. The recommendation could be reached by lifestyle alterations such as recommendation to get more exposure to natural sunlight.

Using the methods and systems of the present invention, the subject could select from these various recommendations from the computer system in order to meet the new recommended daily intake of vitamin D.

1. Method for determining recommended daily intake of nutrients for an individual wherein said method comprises the steps of:
(i) determining the SNP genotype profile of an individual subject from a DNA sample from said subject;
(ii) comparing the SNP genotype profile of an individual subject to a reference SNP genotype profile;
(iii) calculating the individual's genetic risk score per nutrient for at least one of a plurality of nutrients;
(iv) comparing the reference recommended daily intake of said nutrient to the personalised recommended daily intake of said nutrient based on the individual's genetic risk score per nutrient for at least one of a plurality of nutrients; and
(v) providing the personalized recommended daily intake to the individual for at least one of a plurality of nutrients.

2. Method according to claim 1 wherein the personalised recommended daily intake based on the individual's genetic risk score per nutrient is classified as above average, average or below average compared to the reference recommended daily intake per nutrient for at least one of a plurality of nutrients.

3. Method according to claim 1 wherein the SNP genotype profile of an individual subject is determined from a DNA sample from said subject by buccal swab.

4. Method according to claim 1 wherein the at least one of a plurality of nutrients are selected from the group comprising vitamins and/or minerals.

5. Method according to claim 1 wherein the at least one of a plurality of nutrients is Vitamin D.

6. Method according to claim 1 wherein the personalised recommended daily intake for at least one of a plurality of nutrients comprises providing recommendations for nutrient supplements to meet the individual's personal recommended daily intake of at least one of a plurality of nutrients.

7. Method according to claim 1 wherein the personalised recommended daily intake for at least one of a plurality of nutrients comprises providing recommendations for foods, beverages and/or daily meal planning to meet the individual's personal recommended daily intake of at least one of a plurality of nutrients.

8. Computer implemented method according to claim 1 wherein the personalised recommended daily intake for at least one of a plurality of nutrients comprises:
(i) collecting individual user daily consumable nutrient intake data via interaction with a computer user interface;
(ii) calculating the individual user daily consumable nutrient intake data per consumable event at different time points during the day; and
(iii) providing recommendations via a computer user interface for the recommended daily intake per nutrient for the individual user by recommending nutrient supplements, foods, beverages and/or meals for the remaining time during the day to meet the new recommended daily intake for that individual user for at least one of a plurality of nutrients.

9. Computer implemented method according to claim 1 wherein said individual user's recommended daily intake for nutrients is connected to a dispensing device which dispenses nutrient supplements, foods, beverages or complete meals with the recommended daily intake of at least one of a plurality of nutrients that have been personalized for that individual user.

10. A computer implemented system for individualizing recommendation of daily intake of nutrients comprising:
(i) a module with a reference database of recommended daily allowance of nutrients;
(ii) a module with a reference database of SNPs per nutrient;
(iii) a module with individual user attribute data with individual user SNP data per nutrient and a genetic risk score per nutrient;
(iv) a module with a reference database of nutrients per food item;
(v) a module with a reference database of nutrients per beverage item;
(vi) a module with a reference database of nutrient supplements per nutrient;
(vii) a module with a reference menu database;
(viii) a module with a reference recipe database;
(ix) a module with dietary restrictions or individual user preferences; and
(x) a module for optimisation of recommendations
wherein said system provides recommendations for individual user's recommended daily intake per nutrient for at least one of a plurality of nutrients to improve or maintain health in an individual.

11. A computer implemented system for individualizing recommendation of daily intake of nutrients according to claim 10 consisting of:
(i) a module with a reference database of recommended daily allowance of nutrients;
(ii) a module with a reference database of SNPs per nutrient; and
(iii) a module with individual user attribute data with individual user SNP data per nutrient and a genetic risk score per nutrient;
wherein said system provides recommendations for individual user's recommended daily intake per nutrient for at least one of a plurality of nutrients to improve or maintain health in an individual.

12. A computer implemented system according to claim 10 wherein said system comprises recommendations for nutrient supplements to meet the recommended daily intake of at least one of a plurality of nutrients.

13. A computer implemented system according to claim 10 wherein said system comprises recommendations for foods and beverages to meet the recommended daily intake of at least one of a plurality of nutrients.

14. A computer implemented system according to claim 10 wherein said system comprises recommendations for daily meal and menu planning to meet the recommended daily intake of at least one of a plurality of nutrients.

15. A computer implemented system according to claim 10 wherein said system is connected to a dispensing device to dispense the recommended food, beverage, nutrient supplement, or meal.

16. (canceled)
